(19) United States
(12) Patent Application Publication
(10) Pub. No.: US 2010/0255585 A1
(43) Pub. Date: Oct. 7, 2010

(54) PLANT STEM CELL LINE DERIVED FROM QUIESCENT CENTER AND METHOD FOR ISOLATING THE SAME

(75) Inventors: Young-Mi Yu, Jeollabuk-Do (KR); Eun Kyong Lee, Jeollabuk-Do (KR); Sun Mi Hong, Jeollabuk-Do (KR); Young Woo Jin, Jeollabuk-Do (KR)

Correspondence Address: EDWARDS ANGELL PALMER & DODGE LLP, P.O. BOX 55874, BOSTON, MA, 02205, US

(73) Assignee: UNHWA CORPORATION, Jeonju, Jeollabuk-Do (KR)

(21) Appl. No.: 12/679,263

(22) PCT Filed: Sep. 22, 2008
(86) PCT No.: PCT/KR2008/005604

§ 371 Date: Mar. 19, 2010

(30) Foreign Application Priority Data
Sep. 21, 2007 (KR) 10-2007-0096892

(51) Int. Cl. C12N 5/04; C12N 5/02

(52) U.S. Cl. 435/420; 435/410

(57) ABSTRACT

The present invention relates to a cell line derived from the quiescent center of a plant and a method for isolating the same, more specifically, relates to a quiescent center-derived homogeneous cell line of single-cell origin, which is obtained from the quiescent center of a plant without needing a separate de-differentiation process, and a method for isolating the same.

### TECHNICAL FIELD

The present invention relates to a cell line derived from the quiescent center of a plant and a method for isolating the same, and more particularly to a quiescent center-derived homogeneous cell line of single-cell origin, which is obtained from the quiescent center of a plant without a separate de-differentiation process, and a method for isolating the same.

### BACKGROUND ART

Plants have been used as food resources in the past, but the meaning thereof is currently expanded to include a source for a wide range of chemical substances, including drugs, perfumes, pigments, agricultural chemicals and dyes. Particularly, because most useful substances derived from plants have physiological activities, including antiviral, antibacterial, anticancer and antioxidant activities, plants are considered as ideal resources which can be developed into novel drugs, and active studies are in progress to elucidate the relationship between the chemical structures and activities of many plant-derived substances.

However, physiologically active substances are difficult to develop into drugs, and the main reasons therefore are as follows. First, the contents of physiologically active substances in plants are very limited. Second, the growth rate of plants is very slow. Third, physiologically active substances derived from plants are present only in specific organs of plants in small amounts. Fourth, environmental problems associated with the destruction of nature are involved. Fifth, physiologically active substances derived from plants have very complicated chemical structures, such that multi-step polymerization processes are required, thus causing economic problems of high production costs. For these reasons, it was very difficult to stably supply physiologically active substances derived from plants for commercialization.

Meanwhile, a plant cell culture method, one of bioengineering techniques, has been evaluated for a long time as the most ideal technique which can supply plant-derived useful substances without causing environmental problems. According to Korean Patent Publication 1995-0000870, the production of useful substances by plant cell culture technique provides many advantages over methods of extracting useful substances directly from plants. Particularly, the plant cell culture method has been regarded as an optimal method which allows continuous production without being influenced by external environments so as to solve outstanding problems such as ecosystem destruction, unlike the prior extraction methods. However, despite great interest and effort in plant cell culture, examples which succeeded in industrializing the plant cell culture are still insufficient. This is because the variation in cell growth and productivity in a number of plant cell cultures still remains as a major problem.

If plant cells are used in plant expression systems, tissues which differentiated from the plant cells, for example, leaves, stems and seeds, are permanent tissues whose cells no longer divide. For this reason, a de-differentiation process is necessarily carried out in advance in order to convert the tissues into a cell line having the ability to divide. The de-differentiation process means de-differentiating a tissue or cell, which have already been differentiated so as to perform specific functions, when a plant tissue or organ is cultured. However, during this de-differentiation process, serious changes in the cell line can occur due to somaclonal variations.

Particularly, the production of useful substances through plant cell culture can be industrialized, only when rapid cell growth and high metabolite productivity are stably maintained during a long-term culture period. However, most cells undergo many variations due to subculture. Thus, it is urgent to overcome this problem of variations in cells and to develop a method for acquiring a genetically stable cell line in producing useful substances through plant cell culture.

Meanwhile, because plants must absorb sufficient amounts of water or minerals required for the growth thereof, the surface area of the root is significantly large. In the tip of the roots, there are root apical meristem cells, which divide, expand, elongate and differentiate to form the primary root tissue. The root apical meristem is covered by a protective root cap, and the cells in the center of the root apical meristem are called a “quiescent center”, because they divide very slowly. The quiescent center is covered by progenitor cells which make a primary meristem.

However, studies on the physiological characteristics of the root apical meristem are insufficient, even though these characteristics are very important in the root system of plants. Specifically, there were studies in which plants such as maize were used to study the physiological characteristics of the quiescent center or root apical meristems were cultured in media to develop into roots, but there was no example in which, among various tissues of the root system, including root cap, vascular tissue, pericycle, endodermis, cortex and epidermis, only the quiescent center was isolated to establish a cell line.

Thus, the quiescent center is genetically the most stable tissue, and the isolation thereof makes it possible to study the development and genetic origin of plants. Accordingly, there has been a need to develop a method for isolating a homogenous cell line from the quiescent center. In recent years, the field of stem cell biology has newly emerged, and many experiments associated with stem cells, including studies on signals involved in developmental processes, have been conducted. However, in the case of animals, methods for isolating and culturing stem cells have been established early, whereas, in the case of plants, there is little or no study on the isolation of stem cells. Consequently, it is considered that the induction and isolation of cell lines derived from the quiescent center will promote the development of stem cell biology.

Accordingly, the present inventors have made many efforts to develop a method for isolating a quiescent center-derived cell line of single-cell origin and to develop plant cells which can be used in a plant expression system without needing a de-differentiation process. As a result, the present inventors have isolated a quiescent center-derived cell line and found that the isolated cell line undergoes little or no change during long-term culture, can be stably cultured, and shows high cell viability during cryopreservation, thereby completing the present invention.

### SUMMARY OF INVENTION

It is an object of the present invention to provide a quiescent center-derived cell line which can be stably cultured.

Another object of the present invention is to provide a method for isolating a quiescent center-derived cell line without needing a de-differentiation process.

To achieve the above objects, in one aspect, the present invention provides a method for isolating a quiescent center-derived cell line, the method comprising: culturing the quiescent center-containing root tissue of a plant, and then collecting an undifferentiated white tissue from the cultured tissue.

In another aspect, the present invention provides a quiescent center-derived cell line, which is derived from the quiescent center of a plant and has the following characteristics:
(a) it is present as single cells during suspension culture;
(b) it shows a morphological characteristic of nuclei larger than those of cell lines derived from tissues other than the quiescent center;
(c) it is surrounded by mucous materials;
(d) it is stably maintained without morphological changes during long-term culture; and
(e) it shows high viability during cryopreservation.

In still another aspect, the present invention provides a method for preserving a plant cell line, which comprises freezing said cell line derived from the quiescent center of plant roots.

Other features and aspects of the present invention will be apparent from the following detailed description and the appended claims.

### BRIEF DESCRIPTION OF DRAWINGS

FIG. 1A is an optical micrograph of a cell line, which was induced by culturing the quiescent center-containing root tissue of a rice plant, but is in a state before it is isolated from the quiescent root, and FIG. 1B is a photograph of the quiescent center-derived cell line, which was isolated from the tissue and cultured for 4 weeks.

FIG. 2 illustrates optical microscopes of tissues, obtained by culturing the quiescent center-containing root tissue of a rice plant in media containing each of 2,4-D (A), CPA (B), IAA (C), IBA (D), NAA (E) and picloram (F).

FIG. 3 illustrates optical micrographs showing the morphological observation of a cell line derived from root tissues other than the quiescent center of a rice plant (FIG. 3 (a)) and a cell line derived from the quiescent center (FIG. 3(b)).

FIG. 4 illustrates optical micrographs of tissues, obtained by culturing the quiescent center-containing root tissues of a maize plant in media containing each of 2,4-D(A), CPA(B), IAA(C), IBA(D), NAA(E) and picloram (F).

FIG. 5 shows morphological changes in cell lines derived from root tissues other than the quiescent center according to the culture period.

FIG. 6 shows the morphological stabilization of the quiescent center-derived cell line according to the culture period.

FIG. 7A is an optical micrograph (400× magnification) of the quiescent center-derived cell line, and 7B is an optical micrograph (400× magnification) of a root tissue-derived cell line.

FIG. 8 illustrates optical micrographs showing the comparison between the cell line derived from root tissues other than the quiescent center (FIG. 8(a)) and the cell line derived from the quiescent center (FIG. 8(b)).

FIG. 9 is a graphic diagram showing the aggregation rates of a cell line derived from root tissues other than the quiescent center and a cell line derived from the quiescent center.

FIG. 10 illustrates optical micrographs showing the comparison of post-cryopreservation viability between the cell line derived from root tissues other than the quiescent center (FIG. 8(a)) and the cell line derived from the quiescent center (FIG. 8(b)).

FIG. 11 is a graphic diagram showing the post-cryopreservation viabilities of the cell line derived from root tissues other than the quiescent center and the cell line derived from the quiescent center.

### DETAILED DESCRIPTION OF THE INVENTION AND PREFERRED EMBODIMENTS

Unless otherwise defined, all technical and scientific terms used herein have the same meaning as commonly understood by one of ordinary skill in the art. Generally, the definitions of various terms used herein are well known and conventionally used in the art.

In one aspect, the present invention relates to a method for isolating a quiescent center-derived cell line, the method comprising: culturing the quiescent center-containing root tissue of a plant, and then collecting an undifferentiated white tissue from the cultured tissue.

Preferably, the quiescent center-containing root tissue of a plant, which is used in the present invention, is obtained by germinating sterilized plant seeds or is root tissue differentiated from callus derived from a part of the plant. Also, a medium which is used in the culture process may be any cell line induction medium known to those skilled in the art, but the tissue culture is preferably performed in any one medium selected from among N6 medium, MS medium, Gamborg B5 medium, LS medium and KAOM medium. More preferably, the tissue culture is performed in 2,4-D-containing medium. Furthermore, in the present invention, the collection of the quiescent center-derived cell line is preferably carried out after 3-6 weeks of the tissue culture.

As used herein, the term “quiescent center” refers to a group of spherical or disk-shaped single cells consisting of 500-1,000 inactive cells located in the center of the root apical meristem. The cells of this cell group are known to be arrested in the G1 phase of the cell cycle for a long time and to divide at an interval of about 15-20 days. These cells are usually present in an inactive state, and divide when they are injured during excision or by radiation treatment. Specifically, when the plant root penetrates into the soil, the root cap effectively protects the root apical meristem, but because the protection is not perfect, the root apical meristem is sometimes injured during the growth of the root. At this time, the cells in the quiescent center divide to form apical meristems and root caps again. Also, upon exposure to X-rays, cells stop dividing, but the quiescent center cells immediately start to divide to form dividing cells without being influenced by X-rays. In other words, the quiescent center is a place in which genetically stable cells are stored. The quiescent center differentiates into procambium, ground meristem and protoderm, which are the primary meristems of the root.

The quiescent center can be obtained from the root of plants. Preferably, it can be obtained from the root of a plantlet germinated from sterilized seeds or can be obtained from a root tissue differentiated from a callus derived from a part of a plant. Preferably, a explant, obtained by removing the root cap from the root tip and collecting a portion of about 1 mm in thickness from the cut surface, is cultured in cell line induction medium. Before the tissue culture, the collected plant root tissue may be subjected to a sterilization process according to a general method known to those skilled in the art. However, when the root of plantlets germinated from sterilized seeds is used, the collected root tissue may not be subjected to a separate sterilization process. The cell line induction medium may be any medium known to those skilled in the art, and examples thereof include, N6 medium (Chu C. C., Proc. Symp. Plant Tissue Cult., Peking, 43, 1978), MS medium (Murashige T. and Skoog F., Physiol. Plant, 15:473, 1962), Gamborg B5 medium (Gamborg O. L. et al., Exp. Cell Res., 50:151, 1968), LS medium (Linsmaier E. M. and Skoog F., Physiol. Plantarum., 18:100, 1965), KAO M medium (Kao K. N. and Michayluk M. R., Planta. (Berl.), 126:105, 1975), but are not limited thereto.

More preferably, the root tissue is cultured in a medium containing 2,4-D among auxins. Herein, 2,4-D is contained at a concentration of 2 mg/L, and more preferably 2-7 mg/L. Specific culture conditions for cell line induction, culture periods, etc. are determined depending on the kinds or characteristics of plant cells, and the determination of such factors is obvious to those skilled on the art.

In the tissue culture, the morphological difference between a cell line derived from root tissues other than the quiescent center and a cell line derived from the quiescent center was observed. The cell line derived from root tissues other than the quiescent center was heterogeneous and showed local differentiations, but the cell line derived from the quiescent center was homogeneous and showed no local differentiation. Also, when observed visually, the cell line derived from root tissues other than the quiescent center had a yellow color, whereas the cell line derived from the quiescent center had a white color and was surrounded by mucous materials. Namely, the cell line derived from the quiescent center appeared as “undifferentiated white tissue”. The mucous material is regarded as mucigens. The mucigens are known as complex polysaccharides, including sugars, organic acids, vitamins, enzymes and amino acids, which are secreted by cells around the root cap and the epidermal cells of the root.

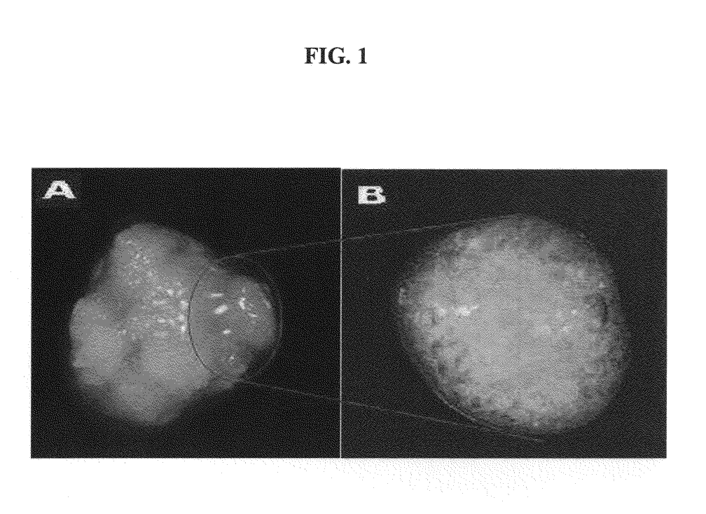

Accordingly, based on this morphological difference, only the quiescent center-derived cell line can be selected. FIG. 1A is a photograph showing the quiescent center-derived cell line before isolation. In FIG. 1A, a red circular portion is the quiescent center-derived cell line. FIG. 1B is a photograph showing the quiescent center-derived cell line, which was isolated and then cultured for 4 weeks.

The collection of the quiescent center-derived cell line is carried out preferably after culture of 3-6 weeks, and more preferably culture of 4-5 weeks after the quiescent center-containing root tissue of a plant is inoculated into a medium. About 3-6 weeks after inoculation, the quiescent center-derived cell line is induced, and thus the isolation thereof becomes easy.

Because the quiescent center is the tissue that all plants have, the inventive method for isolating the quiescent center-derived cell line can be applied to all plants, and thus the quiescent center-derived cell line can be obtained from all plants. Namely, in one Example of the present invention, a cell line was isolated from the quiescent center of the root tissue of rice and maize plants, but it will be obvious to those skilled in the art that the method of the present invention can be applied to all plants having the quiescent center. Examples of plants from which a quiescent center-derived cell line can be obtained may include, but are not limited to, a rice plant, a maize plant, Pisum sativum, Avena sativa, Allium cepa and Arabidopsis. Examples of plants, the physiological characteristics of the quiescent center of which have been studied, may include a maize plant, Arabidopsis, Allium cepa, Avena sativa, Pisum sativum, etc [Maize: Georgina Ponce et al., Plant Cell and Environment, 28:719, 2005; Keni Jiang et al., Development, 130:1429, 2003: Arabidopsis: Noriko Kamiya et al., The Plant Journal, 35:429, 2003; Peter Doerner, Current Biology, 8:R42, 1998; Allium cepa: R. Liso, New Phytol., 110:469, 1998; Avena sativa: F. A. L. Clowes, New Phytol., 129, 1982; Pisum sativum: Peter Doerner, Current Biology, 8:R42, 1998].

In another aspect, the present invention relates to a quiescent center-derived cell line, which is derived from the quiescent center of a plant and has the following characteristics:
(a) it is present as single cells during suspension culture;
(b) it shows a morphological characteristic of nuclei larger than those of cell lines derived from tissues other than the quiescent center;
(c) it is surrounded by mucous materials;
(d) it is stably maintained without morphological changes during long-term culture; and
(e) it shows high viability during cryopreservation.

The quiescent center-derived cell line of the present invention shows morphological characteristics of relatively large nuclei. It was observed that the size of the nuclei was about 2-4 μm, which is larger than those of other cell lines derived from tissues other than the quiescent center.

The quiescent center-derived cell line of the present invention also shows very stable cell growth without morphological changes, even when it is cultured for a long period of time. In one Example of the present invention, it was observed that the quiescent center-derived cell line was stably cultured without morphological changes, even when it was cultured for more than 16 weeks. On the other hand, when the cell line derived from root tissues other than the quiescent center was cultured for a long period of time, several local differentiations were observed in the cell aggregation, and particularly, the development of the adventitious root was distinctly shown.

In addition, plant cells are cultured in the form of a cell aggregation, unlike microbial cells being cultured as single cells. This cell aggregation causes an environmental difference between the inside and the outside of the cell aggregation, thus causing changes in cell growth and the production of useful substances. However, the quiescent center-derived cell line of the present invention has no possibility of such changes, because it is cultured as single cells during suspension culture.

The quiescent center-derived cell line according to the present invention can be used in a plant expression system to stably produce useful substances. Also, the quiescent center-derived cell line of the present invention can be cultured according to plant cell suspension culture method, and a specific culture method can be carried out as known in the art.

In still another aspect, the present invention relates to a method for preserving a plant cell line, which comprises freezing said cell line derived from the quiescent center of plant roots.

Moreover, plant cells showed low viability during cryopreservation, but the quiescent center-derived cell line of the present invention showed a very high viability of more than 85%, when it was cryopreserved according to a conventional cell cryopreservation method. If cell lines can be cryopreserved, it is possible to stably supply raw materials and construct a substantial master cell bank. Thus, the quiescent center-derived cell line of the present invention can be supplied in a long-term and stable manner.

The world is now at war for securing research materials (biological resources), and the preservation and identification of biological resources for developing various new drugs and improving food quality, including human tissue, plant seeds, microorganisms, cells and genes, have become important national properties.

Accordingly, because securing research materials leads to national competitiveness, it is required to construct cell line banks for developing, collecting, preserving and distributing cell lines, which are used as essential materials in studies in the bioscience-related field. Thus, when such plant cell banks are constructed, the supply of research materials can become smooth, and the period of studies employing plant cell lines can be shortened.

### EXAMPLES

Hereinafter, the present invention will be described in further detail with reference to examples. It will be obvious to those skilled in the art that these examples are illustrative purpose only and are not to be construed to limit the scope of the present invention, because these examples can be modified into other various forms.

### Example 1

### Isolation of Cell Line Derived from Quiescent Center of Rice Plant

### 1-1: Preparation of Plant Material

Rice seeds were peeled, surface-sterilized with 70% ethanol for 1 minute, soaked in 2% sodium hypochlorite solution for 1 hour, and then washed once or twice with sterilized water. The washed seeds were sufficiently washed with sterilized water for 30 minutes, and then dried to completely remove moisture.

The dried seeds were seeded in N6 medium (CHU MEDIUM, Chu C. C., Proc. Symp. Plant Tissue Cult., Peking, 43, 1978) and cultured at 25° C. for 5 days, such that they were germinated. The composition of the N6 medium is shown in Table 1 below.

TABLE 1

| Composition | | Concentration | | | |
|---|---|---|---|---|---|
| Macroelements | CaCl₂•2H₂O | 1.13 | mM | 125.33 | mg/L |
| | KH₂PO₄ | 2.94 | mM | 400.00 | mg/L |
| | KNO₃ | 27.99 | mM | 2830.00 | mg/L |
| | MgSO₄•7H₂O | 0.75 | mM | 90.27 | mg/L |
| | (NH₄)₂SO₄ | 3.50 | mM | 463.00 | mg/L |
| Microelements | FeNaEDTA | 0.10 | mM | 36.70 | mg/L |
| | H₃BO₃ | 25.88 | μM | 1.60 | mg/L |
| | KI | 4.81 | μM | 0.80 | mg/L |
| | MnSO₄•4H₂O | 19.70 | μM | 3.33 | mg/L |
| | ZnSO₄•7H₂O | 5.22 | μM | 1.50 | mg/L |
| Vitamins | Glycine | 26.64 | μM | 2.00 | mg/L |
| | Thiamine-HCl | 2.96 | μM | 1.00 | mg/L |
| | Pyridoxine-HCl | 2.43 | μM | 0.50 | mg/L |
| | Nicotinic acid | 4.06 | μM | 0.50 | mg/L |

Then, from the plant germinated for 5-6 days, the quiescent center-containing root tissue was collected. The root cap was removed from the root tip, and a portion of 1 mm in thickness from the cut surface was collected as an explant.

### 1-2: Induction and Isolation of Quiescent Center-Derived Cell Line

(1) The explant collected in Example 1-1 was inoculated into N6 media, containing each of 2 mg/L, 3 mg/L and 4 mg/L of 2,4-D, and N6 media, containing each of other auxin hormones, IAA (indole-3-acetic acid), IBA (indole-3-butyric acid), NAA (1-naphthaleneacetic acid), CPA (p-chlorophenoxyacetic acid) or Picloram (4-amino-3,5,6-trichloropicolinic acid) at the same concentrations as the above.

As a result, in the case of the 2,4-D-containing media, the induction of cell lines was observed 30 days after the inoculation. In the 2,4-D-containing media, the induction rate of the quiescent center-derived cells did not increase in proportion to the concentration thereof and showed similar induction rates at concentration of 2 mg/L and higher. Accordingly, it could be seen that it is preferable to culture the explant at concentration of more than 2 mg/L. In addition, experiments were carried out at concentrations of 1 mg/L, 4 mg/L, 5 mg/L, 6 mg/L and 7 mg/L and, as a result, it was shown that it is more preferable to culture the explant at concentration of 2-7 mg/L.

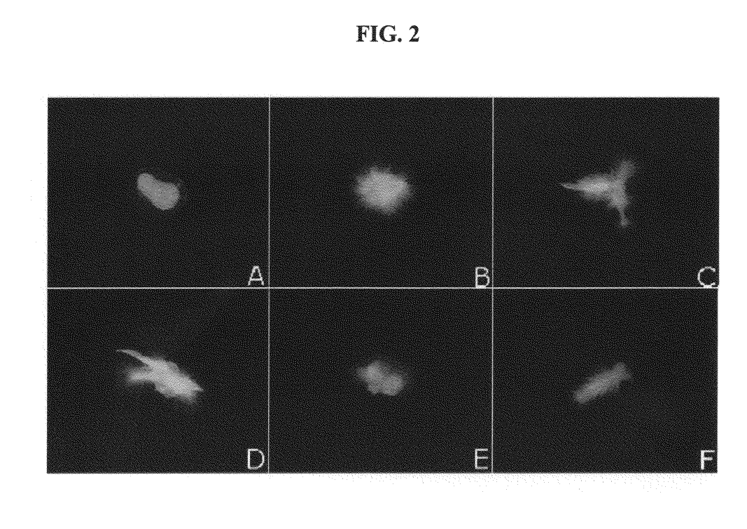

However, as shown in FIG. 2, in the case of auxins other than 2,4-D, cells were not induced after the inoculation, and adventitious roots occurred in the explant. In FIG. 2, “A” is tissue cultured in 2,4-D-containing media, “B” is tissue cultured in CPA-containing media, “C” is tissue cultured in IAA-containing media, “D” is tissue cultured in IBA-containing media, “E” is tissue cultured in NAA-containing media, and “F” is tissue cultured in Picloram-containing media.

Accordingly, it could be seen that the quiescent center-derived cell line was specifically induced, when 2,4-D was used.

(2) As shown in FIG. 1A, when the tissue culture was performed in the 2,4-D-containing media, the morphological difference between the cell line derived from root tissues other than the quiescent center and the cell line derived from the quiescent center was observed. Specifically, the cell line derived from root tissues other than the quiescent center was heterogeneous and showed local differentiations, but the cell line derived from the quiescent center was homogenous and showed no local differentiation. When observed visually, the cell line derived from root tissues other than the quiescent center had a yellow color, whereas the cell line derived from the quiescent center had a white color and was surrounded by mucous substances. Based on this morphological difference, the tissue, which showed no differentiation and had a white color, that is, the cell line derived from the quiescent center, was isolated after 3-6 weeks. In FIG. 1A, a red circular portion is the quiescent center-derived cell line.

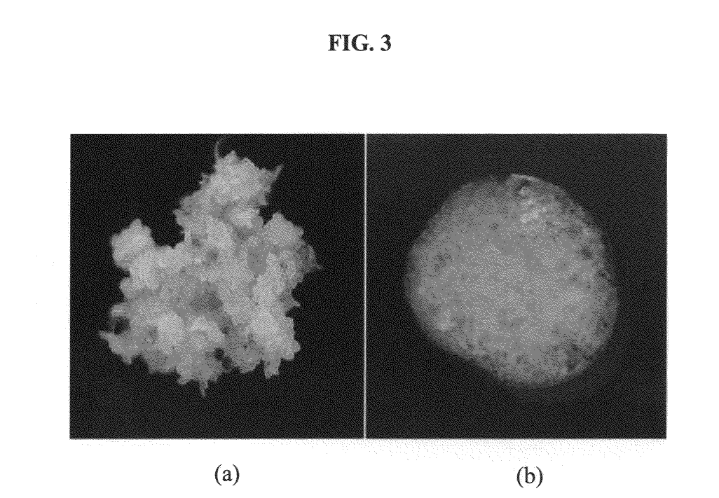

(3) FIG. 3 illustrates photographs showing the morphological observation of the cell line derived from root tissues other than the quiescent center (a) and the cell line derived from the quiescent center (b). As shown in FIG. 3, the cell line derived from root tissues other than the quiescent center was heterogeneous and showed local differentiations, but the cell line derived from the quiescent center was homogenous and showed no local differentiation.

(4) Meanwhile, in the case where the cell line derived from the quiescent center was not isolated even after 3-6 weeks and was mixed with the cell line derived from root tissues other than the quiescent center, the denaturation of the mucous components occurred. This denaturation is believed to be attributable to the phenol compound of the cell line derived from the other tissues. This is because the synthesis of phenol components actively occurs in differentiated tissues.

### Example 2

### Isolation of Cell Line Derived from Quiescent Center of Maize Plant

Maize seeds were germinated in the same manner as in Example 1-1, and from the root of the germinated plant, the quiescent center-containing explant was collected. Then, the explant was cultured in media containing each of 2,4-D, CPA, IAA, IBA, NAA and Picloram, in the same manner as in Example 1-2, and whether the quiescent center-derived cell line was induced was observed.

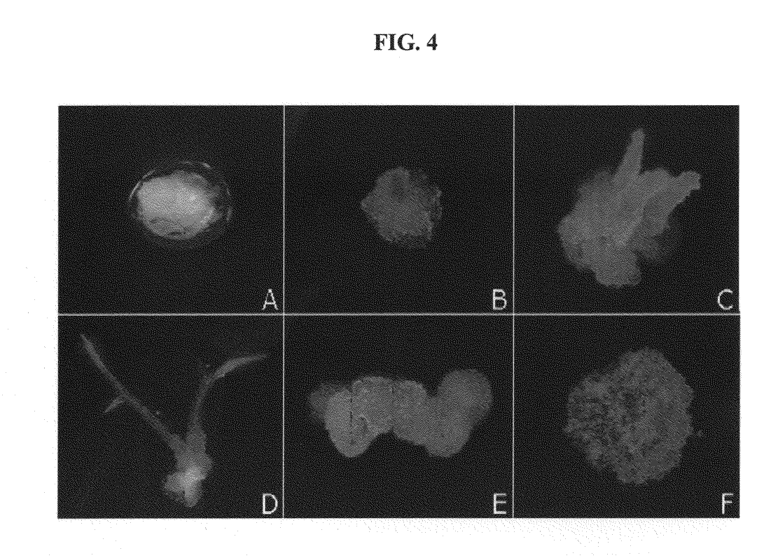

As a result, as shown in FIG. 4, when the quiescent center-containing explant of the maize plant was cultured, the induction of the cell line was observed in the 2,4-D-containing media in the same manner as the case in which the quiescent center-containing root explant of the rice plant was cultured. However, in the case of auxins other than 2,4-D, cells were not induced after the inoculation of the explant, and adventitious roots developed in the explant. In FIG. 4, “A” is a tissue cultured in 2,4-D-containing media, “B” is a tissue cultured in CPA-containing media, “C” is a tissue cultured in IAA-containing media, “D” is a tissue in IBA-containing media, “E” is a tissue cultured in NAA-containing media, and “F” is a tissue cultured in Picloram-containing media.

Accordingly, it could be seen that, even in the case of quiescent center-containing plants other than a rice plant, quiescent center-derived cell lines could be specifically induced, when 2,4-D was used.

### Example 3

### Observation of Characteristics of Cell Line Derived from Quiescent Center of Rice Plant

### 3-1: Observation of Morphological Change During Long-Term Culture

The quiescent center-derived cell line isolated in Example 1 was inoculated into a culture medium having the same composition as in the cell line induction medium of Example 1-2, that is, N6 media containing 2 mg/L of 2,4-D, and was allowed to proliferate. After 4 weeks and 16 weeks of cell growth, the proliferated quiescent center cells were observed. Meanwhile, as control groups, cell lines derived from root tissues other than the quiescent center were allowed to proliferate in the same manner as described above, and then the morphological changes of the cells were observed.

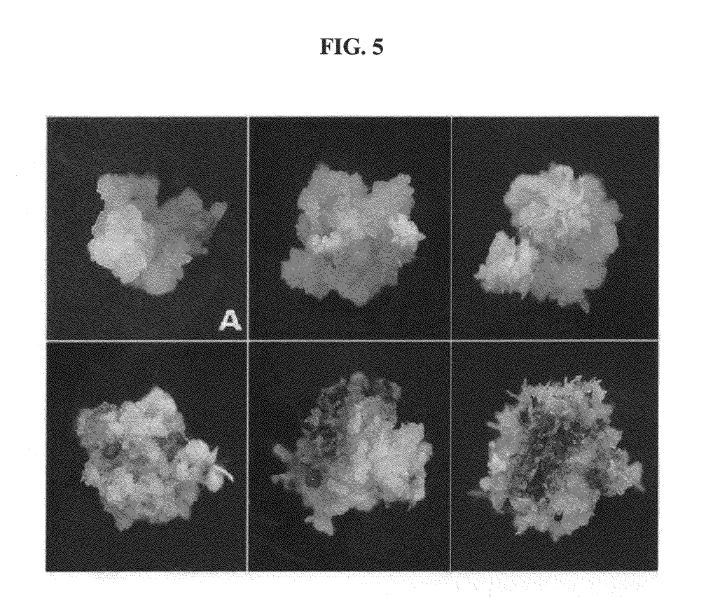

As a result, as shown in FIG. 5, morphological changes were observed in the root tissue-derived cell lines after 4 weeks (FIG. 5A) and 16 weeks (photographs other than FIG. 5A) of culture. After 16 weeks of culture, several local differentiations were observed in the cell aggregation, and particularly, the development of adventitious roots could be distinctly observed.

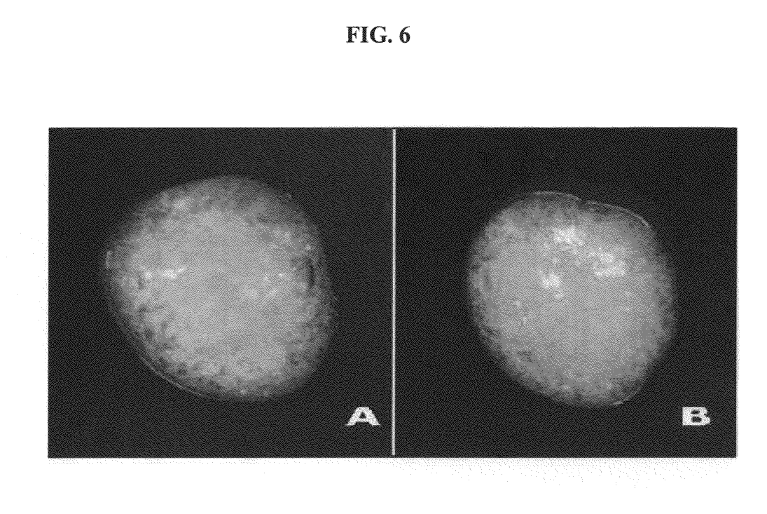

On the other hand, as shown in FIG. 6, when the quiescent center-derived cell line was cultured, no morphological change occurred even after 4 weeks (FIG. 6A) and 16 weeks (FIG. 6B) of culture.

Accordingly, it could be observed that the cell lines derived from root tissues other than the quiescent center showed rapid morphological changes with passage of time, but the quiescent center-derived cell line showed morphological stability. As described above, the quiescent center-derived cell line according to the present invention, which is a single-cell-derived cell line, is stably maintained without changes during long-term culture. Thus, it is very preferably used to select a cell line which has high yield and stable material production ability.

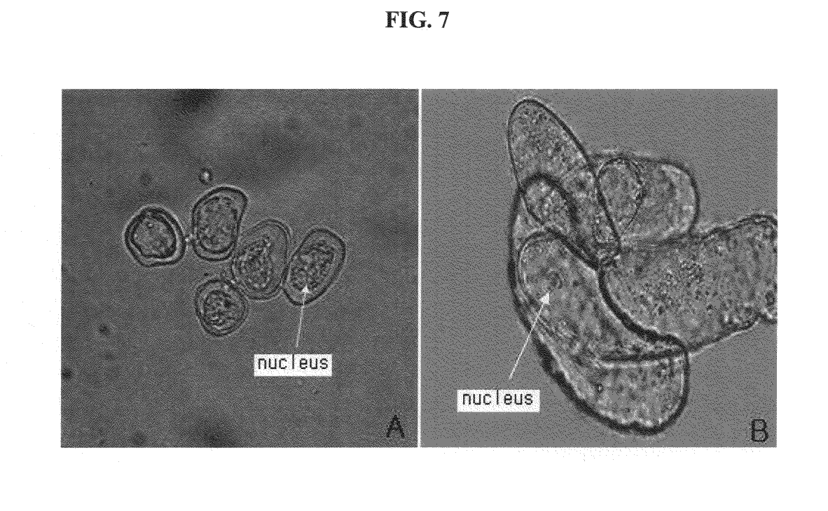

Meanwhile, the isolated quiescent center-derived cell line had morphologically large nuclei. As shown in FIG. 7A, the size of the cell line itself was about 10-20 μm, and the size of the nuclei was about 2-4 μm. On the other hand, as shown in FIG. 7B, the nucleus size of the cell line derived from root tissues other than the quiescent center was very small compared to the quiescent center-derived cell line.

### 3-2: Observation of Cell Aggregation During Suspension Culture

Plant cells are cultured in the form of a cell aggregation without being cultured as single cells, and the cell aggregation consists of a few to several hundreds of plant cells. This cell aggregation causes an environmental difference between the inside and the outside of the cell aggregation, leading to changes in cell growth and the production of useful substances. Accordingly, whether the quiescent center-derived cell line and the cell line derived from root tissues other than the quiescent center aggregate, during suspension culture, was observed.

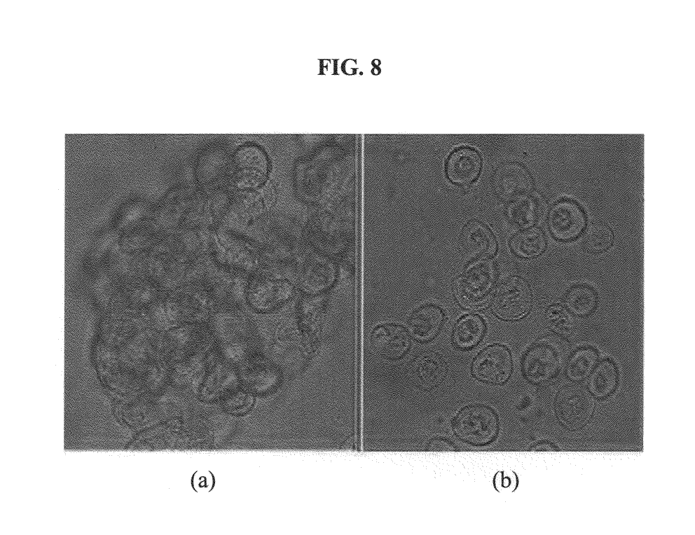

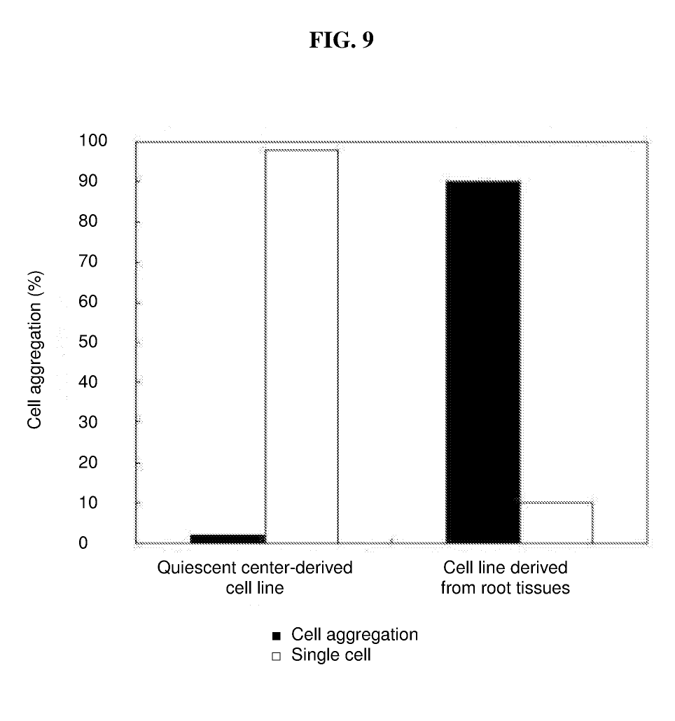

As a result, as shown in FIG. 8 and FIG. 9, the cell line derived from root tissues other than the quiescent center was cultured in a cell aggregation consisting of a few to several hundreds of cells, but the quiescent center-derived cell line was cultured as single cells. FIG. 8 illustrates the cell levels of the cell line derived from root tissues other than the quiescent center (FIG. 8(a)) and the quiescent center-derived cell line (FIG. 8(b)). FIG. 9 is a graphic diagram showing the degree of aggregation of the cell line derived from root tissues other than the quiescent center and the quiescent center-derived cell line.

### 3-3: Experiment of Viability During Cryopreservation

A technique for cryopreserving cell lines is a method essential for supplying raw materials and constructing substantial master cell banks. The cryopreservation technique is commonly widely used in animal cells, but the application thereof in plant cells is limited, because the plant cells have low viability after cryopreservation. Accordingly, the cryopreservation viability of the cell line derived from the quiescent center was tested in the following manner.

The quiescent center-derived cell line of Example 3-2 was inoculated and suspension-cultured for 6-7 days. The suspension culture was pre-cultured in 0.16M mannitol-containing medium at room temperature for 3 days, and then incubated at 4° C. for 3 hours. The low-temperature-treated cells were collected, and the cells were transferred into Cryobial (Duran, USA) containing a medium, containing 40% ethylene glycol (Sigma, USA) and 30% sorbitol (DUCHEFA, The Netherlands), and then were cultured at 4° C. for 3 minutes. Then, the cultured cells treated with a cryopreservation agent were immersed and frozen in liquid nitrogen. Then, for thawing, the cultured cells, which maintained in liquid nitrogen for at least 10 minutes, were placed and maintained in a water bath at 40° C. for 1-2 minutes. For cell regrowth, the filtrate containing the cells was applied to 0.5M sorbitol-containing medium and stabilized at room temperature for 30 minutes. Then, the cells were cultured in 0.1 M sorbitol-containing medium for 24 hours, medium containing no sorbitol for 24 hours, and then medium containing no sorbitol for 24 hours. Then, the viability of the cells was observed.

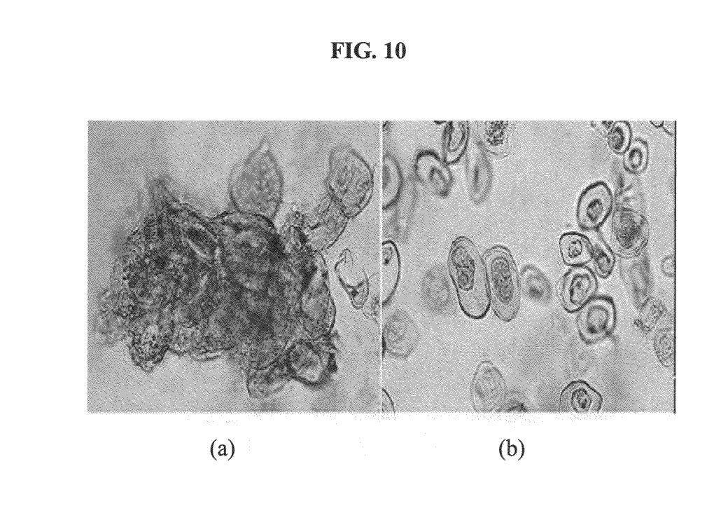

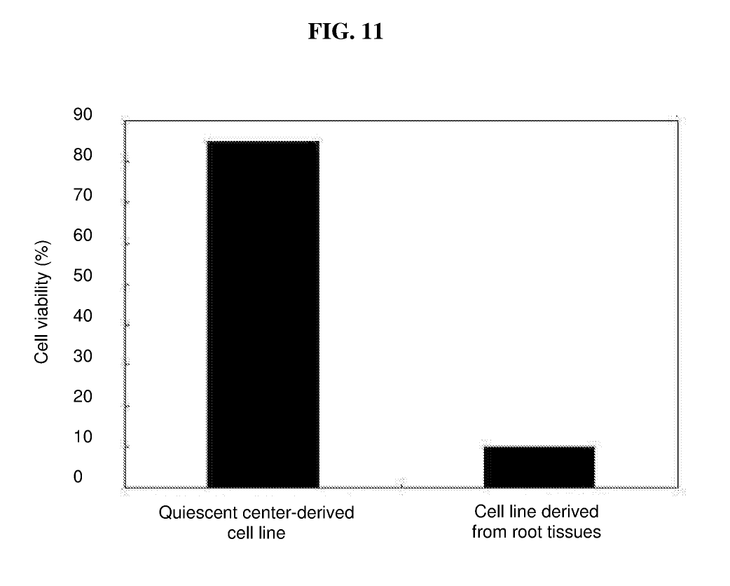

As a result, as shown in FIG. 10 and FIG. 11, the cell line derived from root tissues other than the quiescent center showed a viability of less than 10%, but the quiescent center-derived cell line according to the present invention showed a viability of about more than 85%. FIG. 10 illustrates photographs showing the viabilities of the cell line derived from root tissues other than the quiescent center (a) and the cell line derived from the quiescent center (b), observed through Evan's Blue staining after cryopreservation, and FIG. 11 is a graphic diagram showing the viabilities.

Accordingly, it could be seen that the quiescent center-derived cell line of the present invention could be cryopreserved, unlike conventional plant cells which could not be cryopreserved due to low viability. This suggests that the quiescent center-derived cell line is suitable for long-term preservation.

### INDUSTRIAL APPLICABILITY

As described above, the quiescent center-derived homogeneous cell line isolated according to the method of the present invention is useful as a research tool for studying developmental and genetic origin and assists in the development of plant stem cell biology. Meanwhile, the quiescent center-derived cell line according to the present invention is maintained for a long period of time without morphological changes and cultured as single cells during suspension culture. Accordingly, the cell line allows various useful plant substances to be produced in a safe and effective manner, and makes it possible to construct plant cell banks using a cryopreservation method, because it shows a viability of more than 85% during cryopreservation, unlike other plant cells. Namely, the cell line according to the present invention has advantages in that plant cell banks can be constructed therefrom, thus making it possible to supply research materials smooth, and shorten the period of studies employing plant cell lines.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A method for isolating a quiescent center-derived cell line, the method comprising culturing the quiescent center-containing root tissue of a plant, and then collecting an undifferentiated white tissue from the cultured tissue, wherein said quiescent center-derived cell line has the following characteristics:
(a) it is present as single cells during suspension culture;
(b) it shows a morphological characteristic of nuclei larger than those of cell lines derived from tissues other than the quiescent center;
(c) it is surrounded by mucous materials;
(d) it is stably maintained without morphological changes during long-term culture; and
(e) it shows high viability during cryopreservation.

2. The method for isolating a quiescent center-derived cell line according to claim 1, wherein the quiescent center-containing root tissue of a plant is obtained by germinating sterilized plant seeds or is root tissue differentiated from callus derived from a part of the plant.

3. The method for isolating a quiescent center-derived cell line according to claim 1, wherein the tissue culture is performed in 2,4-D-containing medium.

4. A quiescent center-derived cell line, which is derived from the quiescent center of a plant and has the following characteristics:
(a) it is present as single cells during suspension culture;
(b) it shows a morphological characteristic of nuclei larger than those of cell lines derived from tissues other than the quiescent center;
(c) it is surrounded by mucous materials;
(d) it is stably maintained without morphological changes during long-term culture; and
(e) it shows high viability during cryopreservation.

5. The quiescent center-derived cell line according to claim 4, wherein the plant is selected from the group consisting of a rice plant, a maize plant, Pisum sativum, Avena sativa, Allium cepa and Arabidopsis.

6. A method for preserving a plant cell line, which comprises freezing the cell line derived from the quiescent center of plant roots according to claim 4 or 5.
